(19) United States
(12) Patent Application Publication
(10) Pub. No.: US 2005/0148063 A1
(43) Pub. Date: Jul. 7, 2005

(54) Disposable reaction vessel with integrated optical elements

(76) Inventors: Raymond Francis Cracauer, Middleton, WI (US); Rocky Ganske, Oakville (CA); Adam Brian Liederman, Toronto (CA)

Correspondence Address: Ralph A. Dowell of DOWELL & DOWELL P.C., 2111 Eisenhower Ave., Suite 406, Alexandria, VA, 22314, US

(21) Appl. No.: 10/744,954

(22) Filed: Dec. 24, 2003

(51) Int. Cl. C12M 1/34

(52) U.S. Cl. 435/287.2

(57) ABSTRACT

The present invention provides disposable, semi-reusable, or single use reaction vessels with integrated optical elements for use with diffraction based assay systems. The vessel for assaying liquids for analytes includes a housing having at least one chamber or well for receiving a liquid therein and an optical element integrally formed with the housing for directing an incident light beam towards the well or chamber and directing a light beam away from the chamber after the light beam has interacted with analytes present in the liquid. The vessel may be test tube such as a blood collection tube, with or without, an optical element but having a pattern of analyte-specific receptors located on an inner surface of the tube wall so that when a liquid is introduced into the interior of the test tube analytes present in the liquid can bind with the pattern of analyte-specific receptors.

### FIELD OF THE INVENTION

The present invention relates to disposable, semi-reusable, or single use reaction vessels with integrated optical elements for use with diffraction based assay systems.

### BACKGROUND OF THE INVENTION

With the rapid development of economic, portable and efficient biological assays it has become necessary to be able to rapidly assay large numbers of samples.

In the particular area of optical interrogation of liquid samples using diffraction techniques, one of the difficulties presented in the use of the systems is the need to establish a high quality optical coupling between the reaction substrate and the optics (typically a prism when total internal reflection is used) used to direct the incident beam and the diffracted beams. Any gaps or surface defects on either the prism surface adjacent to the reaction substrate or on the substrate face adjacent to the prism will result, at best, in scattered light which will present as optical noise and thus increased background noise. As is usual with analytical systems, such increased background noise will either limit the sensitivity of detection or will require additional physical or mathematical means to remove the background and thus enhance the detection of the desired signal.

There are several methods currently in use for avoiding these problems. The mating optical surfaces may be manufactured to very high standards of flatness and surface finish. This minimizes the deleterious effects noted, but the cost of providing such surfaces is high and the surfaces are apt to suffer damage in routine use. The most common problem likely to be encountered is scratching of the interface surfaces, particularly the prism.

Both inherent and consequent defects may be mitigated by the use of a refractive index matching fluid on the mating surfaces. Such fluids will fill in small gaps and scratches and minimize scatter created by these defects. However, fluid coupling is problematic. The fluids (eg. silicone fluids and perfluorocarbon fluids) are by their nature messy and difficult to remove since they are highly solvent resistant and cling tenaciously to surfaces. These properties make cleaning of both the optical surfaces and surrounding areas difficult. Additionally, any residual fluid on the prism surface will likely entrain dust particles. These particles will also create scatter in the optical signal and thus increase noise and decrease sensitivity. Further, the requirement to use an interface fluid makes the system less acceptable to users and less amenable to automation of the analytical process.

It would therefore be advantageous to provide an economical and easy to use assay chamber for sample assays that eliminates this requirement.

### SUMMARY OF THE INVENTION

To address the problems described above, the present invention integrates an optical element such as a prism (or other optical element) with the reaction chamber eliminating the interface between the two and thus the associated problems. The cost of the prism integrated reaction chamber is essentially the same as for a simple reaction chamber.

In one aspect of the invention there is provided a vessel for assaying liquids for analytes, comprising:
a housing portion including at least one chamber for receiving a liquid therein; and
at least one optical element integrally formed with the housing portion for directing an incident light beam towards the at least one chamber and directing a light beam away from the at least one chamber after the light beam has interacted with analytes present in the liquid.

In another aspect of the invention there is provided a vessel for assaying liquids for analytes using light diffraction, comprising:
a housing portion including at least one chamber in a top surface thereof for receiving a liquid therein; and
a pre-selected pattern of analyte-specific receptors located on an inner surface of the at least one chamber so that when a liquid is introduced into the interior of the at least one chamber analytes present in the liquid can bind with the pattern of analyte-specific receptors, wherein when analytes bind with the pre-selected pattern of analyte-specific receptors a light beam incident on the pre-selected pattern of analyte-specific receptors is diffracted.

The present invention also provides a test tube, comprising;
a cylindrical tube having a tube wall enclosing an interior and one closed end and one open end for receiving liquid into the interior of the cylindrical tube; and
a pre-selected pattern of analyte-specific receptors located on an inner surface of the tube wall so that when a liquid is introduced into the interior of the test tube analytes present in the liquid can bind with the pattern of analyte-specific receptors.

The present invention also provides a test tube, comprising;
a cylindrical tube having a tube wall enclosing an interior and one closed end and one open end for receiving liquid into the interior of the cylindrical tube;
a pre-selected pattern of analyte-specific receptors located on an inner surface of the tube wall so that when a liquid is introduced into the interior of the test tube analytes present in the liquid can bind with the pattern of analyte-specific receptors; and
at least one optical element integrally formed with the test tube wall for directing an incident light beam towards the at least one chamber and directing a light beam away from the at least one chamber after the light beam has interacted with analytes present in the liquid.

### BRIEF DESCRIPTION OF THE DRAWINGS

The following is a description, by way of example only, of disposable reaction vessels with integrated optical elements constructed in accordance with the present invention, reference being had to the accompanying drawings, in which:

FIG. 1 is a perspective view of a disposable reaction vessel with an integrated optical element having an analyte-specific pattern in a single reaction chamber with a prism integrally formed with the bottom of the reaction chamber;

FIG. 2 is a perspective view of another embodiment of a disposable reaction vessel having an elongated reaction chamber with a linear array of analyte-specific patterns along the bottom of the reaction chamber with an elongated prism integrally formed along the bottom of the housing containing the reaction chamber;

FIG. 3a is a side view of another embodiment of a disposable reaction vessel having a standard micro titer plate with multiple individual solution wells with an individual prism integrally formed along the bottom of each well;

FIG. 3b is a top view of the disposable reaction vessel of FIG. 3a;

FIG. 4 is a top view of another embodiment of a disposable reaction vessel constructed in accordance with the present invention;

FIG. 5(a) shows a top view of another embodiment of a disposable reaction chamber with a micro fluidic channel that carries sample from receptor spot to spot;

FIG. 5(b) shows a side view taken along arrow b of FIG. 5(a);

FIG. 5(c) shows a side view of the high density array with the alternative prism configurations taken along arrow c of FIG. 5(a); and

FIG. 6 shows a test tube having a pattern of analyte-specific receptors formed on an interior surface thereof.

### DETAILED DESCRIPTION OF THE INVENTION

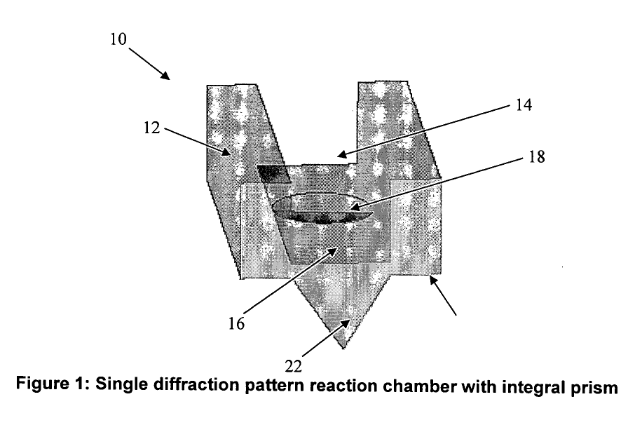

A number of embodiments of the present invention are desirable for differing applications. In one embodiment, a single reaction chamber with integral prism is useful for compact devices requiring assay of one or two analytes. FIG. 1 shows such an embodiment of a disposable reaction vessel 10 with integrated optical element. Reaction vessel 10 includes a housing 12 enclosing a well or chamber 14. Housing 12 has an inner bottom surface 16 on which a pre-selected pattern 18 of analyte receptors is formed for detecting any number of analytes. On an outer bottom surface 20 of housing 12 is a prism 22 which is integrally formed with the rest of housing 12. The housing 12 with integrated prism 22 may be produced of any suitable plastic, generally a clear transparent plastic at the wavelengths to be used to illuminate the pattern through the prism 22.

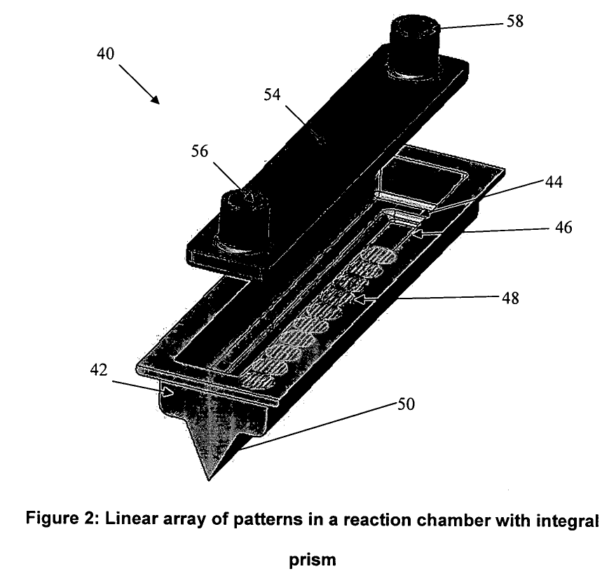

For multiple assay formats using multiple analyte specific patterns but one reaction chamber, the present invention is embodied by disposable reaction vessel 40 shown in FIG. 2 which includes a housing portion 42 enclosing a well or chamber 44 with the housing having an inner bottom surface 46 along which a linear array of analyte specific patterns 48 are formed with an elongated single prism 50 integrally formed along the bottom outer surface of housing 42 thus giving a single consumable with an elongated prism. Disposable reaction vessel 40 includes a housing cover 54 having a fluid inlet 56 and a fluid outlet 58. When housing 42 is assembled with cover 54, fluid containing the analyte to be analyzed may be flowed through inlet 56 and out through outlet 58. In one embodiment, when cover 54 is assembled with housing 42, the volume of interior chamber 44 is such that a capillary flow path is formed through the chamber between the inlet 56 and outlet 58. This embodiment of the disposable reaction vessel 40 with integrated optical elements is appropriate for situations where a compact consumable is desired and up to approximately thirty (30) discrete assays are required.

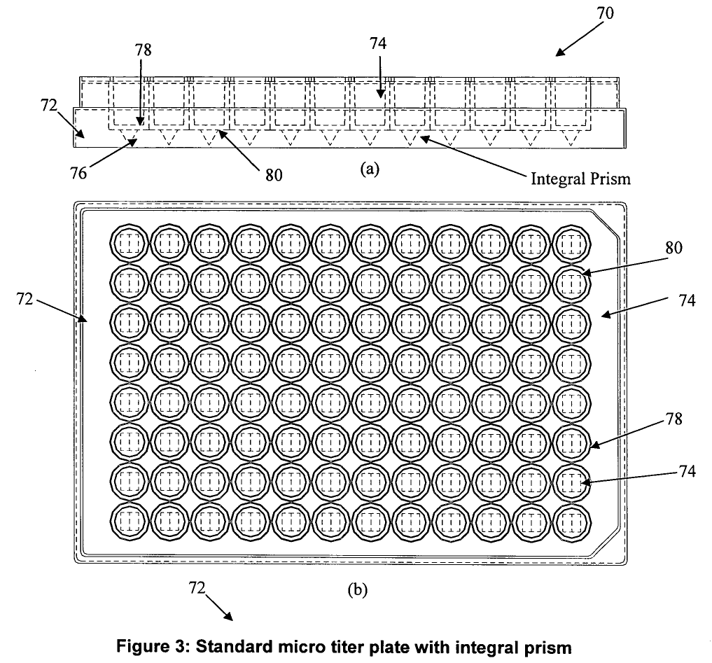

Referring to FIG. 3, another embodiment of a disposable reaction vessel with integrated optical elements is shown generally at 70. This disposable reaction vessel 70 generally reflects the format of a standard micro-titer plate 72, having an array of individual reaction wells 74 each for holding a separate solution. In disposable reaction vessel 70, prisms 76 are molded at the bottom of each reaction well 74 in an array format similar to a standard micro titer plate. Analyte specific patterns 78 are formed on the bottom surface 80 of each reaction well. Disposable reaction vessel 70 has the advantage of being compatible with standard laboratory fluid handling devices (e.g. Tecan, Beckman, or Hamilton laboratory robots) and providing for either large numbers of distinct assays or performing the same assay on a multiplicity of samples or combinations thereof. Therefore disposable reaction vessel 70 would be appropriate for conducting from 96 through 1536 reactions, though extension to higher or lower densities is certainly possible.

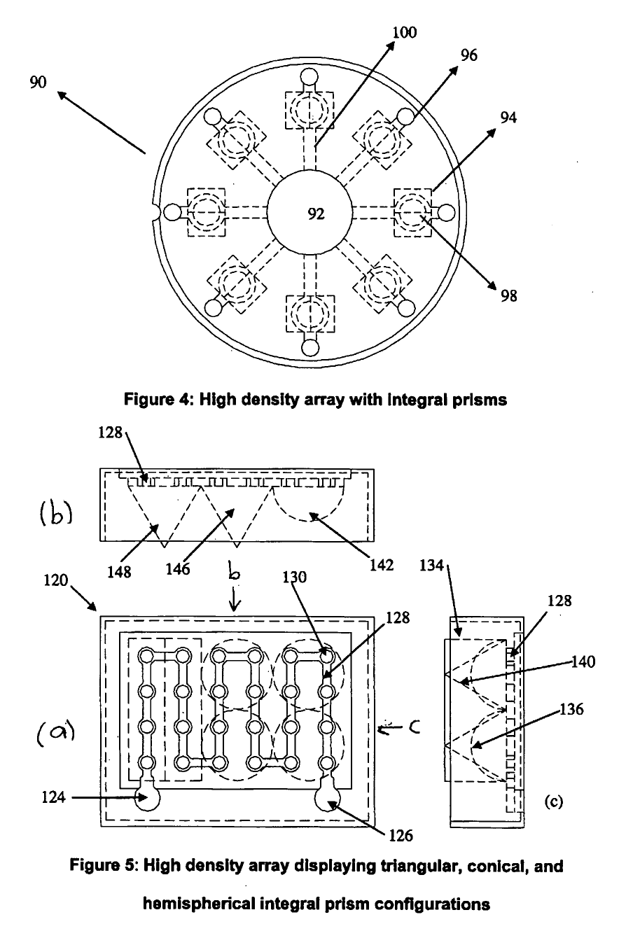

Referring to FIG. 4, another embodiment of a disposable reaction vessel with integrated optical elements is shown generally at 90 and includes a high density array, created in a format which allows large numbers of assays to be conducted on a single sample. Disposable reaction vessel 90 includes a central well 92 in which a sample is introduced. The sample is wicked from the sample well 92 outwardly to the individual wells 94 through the capillary channel 100, by capillary action. The bottom of each well 94 is patterned with a pre-selected pattern of analyte-specific receptor molecules 98. The hole 96 located at the end of each capillary channel 100 allows air to escape from the capillary tube when the sample is introduced to the sample well 92 and wicks through the capillary tube 100. The disposable reaction vessel 90 includes a prism 102 located below each site patterned with the analyte-specific receptors 98. Disposable reaction vessel 90 may be used in a spinning mode in cases where only one optical source-detector system is used. That is, the reaction vessel 90 may be rotated such that the optical elements associated with each reaction site are presented to the excitation and detection optics of a detection instrument. Depending on the mode of operation and details of the associated instrument, the reaction vessel may stop to allow reading or the reading may be taken “on the fly” while the vessel is rotating.

The optical element configuration illustrated in the Figures is shown for convenience in a conventional triangular shape, but those skilled in the art will appreciate that alternative designs may be used to optimize light path and manufacturability.

FIG. 5(a) shows a top view of a high density array with micro fluidic channels that carry liquid sample from receptor spot to spot. FIGS. 5(b) and 5(c) display the use of triangular 148, conical 146, and hemispheric 142 optical elements to direct incident light to the pattern and diffracted light to the detector. FIG. 5(b) shows the front view of the high density array 120 with the front view of the triangular prism 148, conical prism 146, and hemispherical prism 142 in clear view. Sample is introduced to the sample input well 124 and wicks through the sample channel 128 pulled through by capillary action. The sample is pulled through the sample channel 128, across a number of regions patterned with receptor molecules 130, and out the sample output port 126. FIG. 5(b) also shows the front view of the sample channel 128. FIG. 5(c) shows the side view of the high density array 120, displaying the side view of the triangular prism 134, conical prism 140, and the hemispherical prism 136. In this view the depth of the sample channel 128 can be seen.

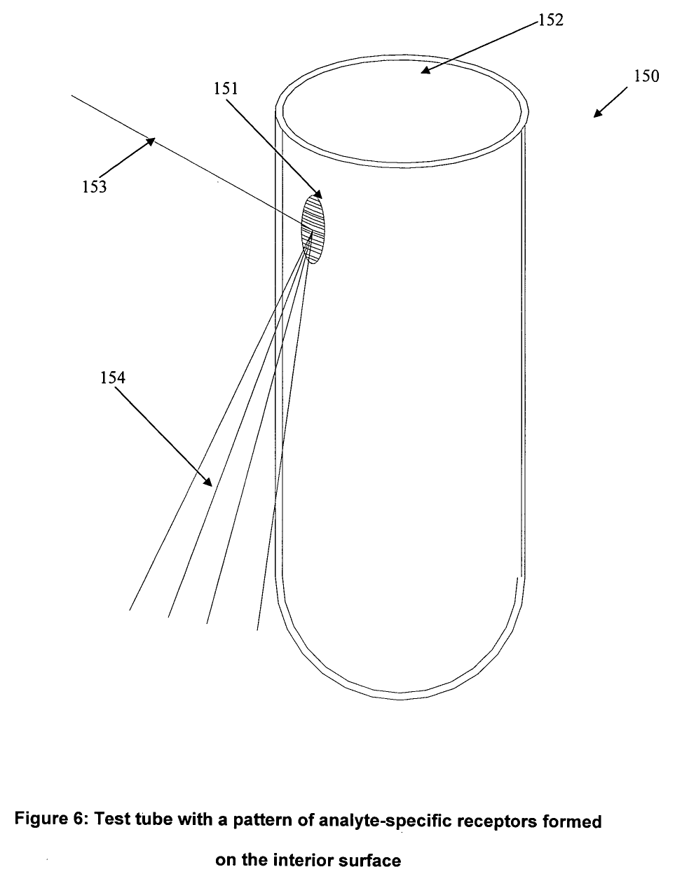

FIG. 6 shows a test tube 150 having a pattern of analyte-specific receptors 151 formed on an interior surface 152 thereof. The incedendent laser beam 153 is seen approaching the analyte-specific receptors 151 with the diffracted laser beams 154 shown moving away from the analyte-specific receptors 151. The sample will be introduced to the test tube 150 up to the level of the analyte-specific receptors 151 and placed in a reader device in order to carry out analysis. The test tube may be a blood collection tube such as typically used in collecting patients' blood. The test tube or blood tube may contain integrated optics adapted to more easily interface the tube with the reader optics.

The pre-selected pattern of analyte-specific receptors located on the inner surface, preferably the bottom of chamber, may be produced using the micro-stamping apparatus described in copending U.S. patent application Ser. No. entitled METHOD AND APPARATUS FOR MICRO-CONTACT PRINTING filed concurrently with the present patent application, the contents of which are incorporated herein in its entirety. The patterns may be regular equi-spaced parallel lines or they may be more complicated patterns as disclosed in copending U.S. patent applications Ser. Nos. 09/814,161 and 10/242,778, both of which are incorporated by reference herein in their entirety.

As used herein, the terms “comprises”, “comprising”, “including” and “includes” are to be construed as being inclusive and open ended, and not exclusive. Specifically, when used in this specification including claims, the terms “comprises”, “comprising”, “including” and “includes” and variations thereof mean the specified features, steps or components are included. These terms are not to be interpreted to exclude the presence of other features, steps or components.

The foregoing description of the preferred embodiments of the invention has been presented to illustrate the principles of the invention and not to limit the invention to the particular embodiment illustrated. It is intended that the scope of the invention be defined by all of the embodiments encompassed within the following claims and their equivalents.

1. A vessel for assaying liquids for analytes, comprising:
a housing portion including at least one chamber for receiving a liquid therein; and
at least one optical element integrally formed with the housing portion for directing an incident light beam towards the at least one chamber and directing a light beam away from the at least one chamber after the light beam has interacted with analytes present in the liquid.

2. The vessel according to claim 1 including a pre-selected pattern of analyte-specific receptors located on an inner surface of the at least one chamber so that when a liquid is introduced into the chamber analytes present in the liquid can bind with the pattern of analyte-specific receptors, and wherein the light beam that has interacted with the pre-selected pattern of analyte-specific receptors and analytes bound thereto is a diffracted light beam.

3. The vessel according to claim 1 wherein the housing portion having at least one chamber is a standard micro-titer plate having ninety-six (96) chambers.

4. The vessel according to claim 3 including a pre-selected pattern of analyte-specific receptors located on an inner surface of the ninety-six (96) chambers so that when a liquid is introduced into the chamber analytes present in the liquid can bind with the pattern of analyte-specific receptors.

5. The vessel according to claim 1 wherein the optical element integrally formed with the housing portion is a triangular shaped optical element.

6. The vessel according to claim 2 wherein the optical element integrally formed with the housing portion is a triangular shaped optical element located below the at least one chamber, and wherein the pre-selected pattern of analyte-specific receptors are located on a bottom surface of the at least one chamber.

7. The vessel according to claim 3 wherein the ninety-six (96) chambers are arranged in rows and columns, and wherein the optical element integrally formed with the housing portion is an elongate triangular shaped optical element located below each column or row of chambers so that a total number of elongate triangular shaped optical elements is equal to the number of columns or rows in the vessel.

8. The vessel according to claim 7 including a pre-selected pattern of analyte-specific receptors located on a bottom surface of each of the ninety-six (96) chambers so that when a liquid is introduced into the chamber analytes present in the liquid can bind with the pattern of analyte-specific receptors, and wherein the light beam that has interacted with material and is a diffracted light beam.

9. The vessel according to claim 2 wherein the optical element integrally formed with the housing portion is a hemispherical-shaped optical element located below the at least one chamber, and wherein the pre-selected pattern of analyte-specific receptors are located on a bottom surface of the at least one chamber.

10. The vessel according to claim 2 wherein the optical element integrally formed with the housing portion is a conically shaped optical element located below the at least one chamber, and wherein the pre-selected pattern of analyte-specific receptors are located on a bottom surface of the at least one chamber.

11. The vessel according to claim 1 wherein the housing portion having at least one chamber includes an array of chambers for holding a plurality of liquid samples separate from each other.

12. The vessel according to claim 1 wherein the housing includes an elongate housing section and wherein the at least one chamber is an elongate chamber defined by the elongate housing section, and wherein the housing includes a cover section having a liquid inlet and a liquid outlet, which, when assembled with the elongate housing section produces a capillary flow path between the liquid inlet and liquid outlet through the elongate housing section.

13. The vessel according to claim 12 including at least one pre-selected pattern of analyte-specific receptors located along a bottom of the elongate chamber so that when a liquid is introduced into the chamber analytes present in the liquid can bind with the at least one pattern of analyte-specific receptors, and wherein the light beam that has interacted with material and substrate is a diffracted light beam.

14. The vessel according to claim 13 wherein the optical element integrally formed with the substrate is an elongate triangular shaped optical element located below the elongate chamber extending along a length of the elongate chamber.

15. The vessel according to claim 1 wherein the housing includes a generally circular substrate, and wherein the at least one chamber for receiving a liquid therein is a first chamber disposed in a center of the circular substrate, including a plurality of chambers radially displaced from the first chamber with each of the plurality of chambers being in flow communication with the first chamber through an associated flow passageway connecting each of the plurality of chambers with the first chamber, and wherein the at least one optical element includes an associated optical element located below each of the plurality of chambers.

16. The vessel according to claim 15 including a pre-selected pattern of analyte-specific receptors located on a bottom surface of each of the plurality of chambers so that when a liquid is introduced into the chamber analytes present in the liquid can bind with the pattern of analyte-specific receptors, and wherein the light beam that has interacted with material and substrate is a diffracted light beam.

17. The vessel according to claim 15 wherein the housing includes a mount for mounting the vessel on a rotational drive mechanism for spinning the vessel.

18. The vessel according to claim 1 made of molded plastic.

19. The vessel according to claim 2 wherein the at least one optical element integrally formed with the housing is located with respect to the inner surface on which the pre-selected pattern is present in order so that light directed by the at least one optical element undergoes total internal reflection.

20. The vessel according to claim 8 wherein the elongate triangular shaped optical elements are located with respect to the bottom surface of the chambers of the associated row of chambers so that light directed by the elongate triangular shaped optical elements undergoes total internal reflection.

21. The vessel according to claim 1 wherein the optical element integrally formed with the housing portion is a hemispherical-shaped optical element.

22. The vessel according to claim 1 wherein the optical element integrally formed with the housing portion is a conical-shaped optical element.

23. A vessel for assaying liquids for analytes using light diffraction, comprising:
a housing portion including at least one chamber in a top surface thereof for receiving a liquid therein; and
a pre-selected pattern of analyte-specific receptors located on an inner surface of the at least one chamber so that when a liquid is introduced into the interior of the at least one chamber analytes present in the liquid can bind with the pattern of analyte-specific receptors, wherein when analytes bind with the pre-selected pattern of analyte-specific receptors a light beam incident on the pre-selected pattern of analyte-specific receptors is diffracted.

24. The vessel according to claim 23 including an optical element integrally formed with the housing portion for directing an incident light beam towards the well and directing a diffracted light beam away from the well after the light beam has interacted with the material and substrate.

25. The vessel according to claim 23 wherein the housing portion having at least one chamber is a standard micro-titer plate having ninety-six (96) chambers.

26. The vessel according to claim 23 including an optical element integrally formed with the housing portion located below each of the ninety-six (96) chambers for directing an incident light beam towards each chamber and directing a diffracted light beam away from the chamber after the light beam has interacted with the pre-selected pattern of analyte-specific receptors and analytes bound thereto.

27. The vessel according to claim 26 wherein each of the ninety-six (96) chambers includes a pre-selected pattern of analyte-specific receptors located on a bottom surface of the at least one chamber.

28. The vessel according to claim 27 wherein the ninety-six (96) chambers are arranged in rows and columns, and wherein the optical element integrally formed with the substrate is an elongate triangular shaped optical element located below each column or row of chambers so that a total number of elongate triangular shaped optical elements is equal to the number of columns or rows in the vessel.

29. A test tube, comprising;
a cylindrical tube having a tube wall enclosing an interior and one closed end and one open end for receiving liquid into the interior of the cylindrical tube; and
a pre-selected pattern of analyte-specific receptors located on an inner surface of the tube wall so that when a liquid is introduced into the interior of the test tube analytes present in the liquid can bind with the pattern of analyte-specific receptors.

30. The test tube according to claim 29 where the test tube is a blood collection tube.

31. The test tube according to claim 29 where the test tube includes an optical element integrally formed therewith for directing an incident light beam towards the interior of the test tube and directing a light beam away from the interior of the test tube.

32. A test tube, comprising;
a cylindrical tube having a tube wall enclosing an interior and one closed end and one open end for receiving liquid into the interior of the cylindrical tube;
a pre-selected pattern of analyte-specific receptors located on an inner surface of the tube wall so that when a liquid is introduced into the interior of the test tube analytes present in the liquid can bind with the pattern of analyte-specific receptors; and
at least one optical element integrally formed with the test tube wall for directing an incident light beam towards the at least one chamber and directing a light beam away from the at least one chamber after the light beam has interacted with analytes present in the liquid.

33. The test tube according to claim 32 where the test tube is a blood collection tube.
